(19) United States
(12) Patent Application Publication
(10) Pub. No.: US 2016/0038736 A1
(43) Pub. Date: Feb. 11, 2016

(54) Electro catheter for neurostimulation

(71) Applicants: Claudio Reverberi, Mirandola (MO) (IT); Eugenio Centin Snichelotto, Rubano (PD) (IT); Enrico Celegato, Rubano (PD) (IT)

(72) Inventors: Claudio Reverberi, Mirandola (MO) (IT); Eugenio Centin Snichelotto, Rubano (PD) (IT); Enrico Celegato, Rubano (PD) (IT)

(73) Assignees: NEURIMPLUSE SRL, Rubano (PD) (IT); CARDIAC IMPULSE SRL, Rubano (PD) (IT)

(21) Appl. No.: 14/366,608

(22) PCT Filed: Dec. 6, 2011
(86) PCT No.: PCT/IB2011/055483

§ 371 Date: Sep. 28, 2015

(30) Foreign Application Priority Data
Dec. 5, 2011 (IT) PD2011A000383

(51) Int. Cl. A61N 1/05

(52) U.S. Cl. CPC A61N 1/0558

(57) ABSTRACT

An electro catheter for neurostimulation includes a stimulation end of essentially helical shape, suited to be wrapped around a nerve. The stimulation end is deformable, meaning that it has a helical shape that can be modified in diameter and/or pitch, according to the dimensions of the nerve to be wrapped, and can be modified even to the point of assuming an essentially rectilinear linear shape according to the profile of the nerve, so that it is suited to be positioned in proximity to the nerve, along its profile.

### FIELD OF THE INVENTION

The present invention concerns implantable devices for antalgic neurostimulation, and in particular concerns a new electro catheter for neurostimulation with adaptable form factor and removable anchorage means. More specifically, the present invention concerns a multipolar electro catheter whose stimulation end can assume a helical or linear shape such to envelop the nerve with the stimulation electrodes.

### BACKGROUND OF THE INVENTION

Neurostimulation has been successfully used for years in the treatment of chronic pain. Antalgic stimulation allows, for example, relieving if not even neutralizing chronic pain through the electrical stimulation of specific nerve fibers. Clinical cases relating to the stimulation of the femoral, tibial, ulnar, sciatic, occipital (greater and lesser), peroneal, radial nerve show the effectiveness of neurostimulation for pathologies like cervical pain, foot causalgia, migraine, carpal tunnel and pain deriving from surgical failures.

The effect perceived by the patient is a feeling of “numbness” that replaces the feeling of pain.

At present, in the field of neurostimulation systems, two families of devices are typically used: the neuro stimulator and the electro catheter.

The neurostimulator is essentially an electronic circuit, powered by means of a battery or a rechargeable accumulator, enclosed within a tight container made of a biocompatible material, typically titanium.

The electronic circuit placed inside the neurostimulator is telemetrically programmable and is a generator of multi-channel electric pulses with programmable duration, frequency and amplitude.

The electro catheter is the means, through which the electric pulses generated by the neurostimulator are carried to the nerves. Electro catheters are usually provided on one side with a series of stimulation electrodes, which are to be positioned in proximity to the nerve to be stimulated, and on the other side with a series of contact electrodes, equal in number to the stimulation electrodes, which are to be connected to the neurostimulator through a suitable connector.

The effectiveness of stimulation essentially depends on the distance between the stimulation electrodes and the nerve: the shorter the distance, the lesser the energy that the electro stimulator has to supply to give the patient the same relief.

Taking in consideration the fact that in the field of neurostimulation implants are usually made by both anesthesiologists, without open surgery, and by neurosurgeons, through open surgery, there is the need to have a multipolar electro catheter capable of meeting the needs of both implanting procedures and of guaranteeing, at the same time, that the stimulation electrodes are positioned as near the nerve as possible.

### SUMMARY OF THE INVENTION

The subject of the present invention is a new percutaneous/neurosurgical electro catheter with a modifiable form factor, designed to satisfy the above described needs.

The main object of the present invention is to provide an electro catheter with modifiable form factor, meaning that its shape can be adapted to nerves having different diameters according to need, in order to guarantee better adherence of the stimulation electrodes to the nerve.

Another object of the present invention is to provide a catheter that can be implanted through a percutaneous technique, by means of a suitable introducer, or through a surgical technique, so that it can be used by both surgeons and anesthesiologists, however guaranteeing maximum effectiveness.

It is another object of the present invention to guarantee effective stimulation by reducing energy consumption by the stimulator connected to the electro catheter.

It is a further object of the present invention to guarantee that the catheter is positioned in a stable manner on the patient's nerve, with consequent long-lasting stability of both stimulation and its effectiveness.

These and other direct and complementary objects are achieved by an electro catheter for neurostimulation according to the invention with adaptable form factor, comprising a stimulation end provided with stimulation electrodes that is essentially helical in shape and is suited to be wrapped around a nerve, wherein said stimulation end has a modifiable shape and can assume the shape of a helix with any pitch and diameter, depending on the dimensions of the nerve around which it is applied, or an essentially rectilinear linear shape, depending on the profile of the nerve along which it is applied.

Said deformation can be obtained, for example, through traction or compression.

If the doctor, that is, the neurosurgeon, decides to proceed with surgery, said electro catheter with helical stimulation end is suited to be wrapped around the nerve.

Said helical shape can be modified so that said end, provided with the stimulation electrodes, comes to assume a helical form factor, with diameter and pitch depending on the dimension of the diameter of the nerve to be wrapped.

For example, taking in consideration that nerves are essentially cylindrical with essentially circular cross section and diameter that can range from 0.5 cm to 3 cm, as in the case of the sciatic nerve, for example, said electro catheter with helical form factor can be adapted to any type of nerve.

Furthermore, said stimulation end, if tightened by exerting a traction force on it, becomes tight around the nerve, adhering to the nerve completely and to the maximum degree.

Owing to the above, the stimulation electrodes of the electro catheter installed on the nerve are always in contact with the nerve, independently of its diameter, thus guaranteeing more effective stimulation with consequent reduced energy consumption by the neurostimulator connected to the electro catheter.

Vice versa, if the doctor, be he/she an anesthesiologist or a neurosurgeon, decides to choose the percutaneous procedure, said electro catheter is also suited to be positioned in proximity to the nerve by means of a suitable introducer, and in this case the correct positioning will be checked using a non invasive procedure.

In this case, the stimulation end of said electro catheter will be deformed until it assumes an essentially rectilinear linear shape, suited to follow the profile of the nerve.

The percutaneous/neurosurgical electro catheter with adaptable form factor, helical in shape and with any pitch or diameter, which can change shape until obtaining even a linear shape, is furthermore provided with one or more preferably removable anchorage means.

Said anchorage means comprise one, two or more pairs of fixing tabs, preferably made of a biocompatible material, suited to be used by the doctor who carries out the implant in order to guarantee a stable position of the electro catheter with respect to the nerve.

Said fixing tabs can be constrained to the tissue around the nerve using a normal suture thread, thus fixing the catheter to the nerve in the desired position.

According to the invention, one of said anchorage means is positioned on the electro catheter before the segment where the stimulation electrodes are positioned, while a second anchorage means is positioned at the end of the same segment.

Said anchorage means are preferably removable, or only said fixing tabs are removable, as their use is fundamentally connected to the implantation technique. While the neurosurgeon can use said anchorage means in open surgery, in the case of a percutaneous procedure the doctor making the implant can remove said anchorage means or said fixing tabs and position the electro catheter under the skin using the suitable introducer.

### BRIEF DESCRIPTION OF THE DRAWINGS

The characteristics of the new electro catheter will be highlighted in greater detail in the following description with reference to the enclosed drawings that are provided by way of examples without limitation.

FIGS. 1a, 1b and 1c respectively show two views and a cross section of AN electro catheter (1) according to the invention with helical stimulation end (2) wrapped around a nerve (N).

FIGS. 2a and 2b show two views of the new electro catheter (1) with helical stimulation end (2) wrapped around a nerve (N) according to a different embodiment of the invention.

FIGS. 3 a, 3b and 3 c respectively show a three-dimensional view and two side views of an anchorage means (3) of the new electro catheter.

FIGS. 4a and 4b respectively show tables and charts regarding the data related to the helix of the stimulation end (2) according to the dimensions of the nerve (N).

### DETAILED DESCRIPTION OF EMBODIMENTS OF THE INVENTION

The invention relates to an electro catheter (1) for neurostimulation, preferably of the multipolar type, comprising a stimulation end (2) provided with one or more stimulation electrodes (4) distributed along at least one segment (21) of said stimulation end (2).

Said electro catheter (1), and in particular said stimulation end (2), is suited to be placed in proximity to a nerve (N) in order to subject it to neurostimulation for pain treatment.

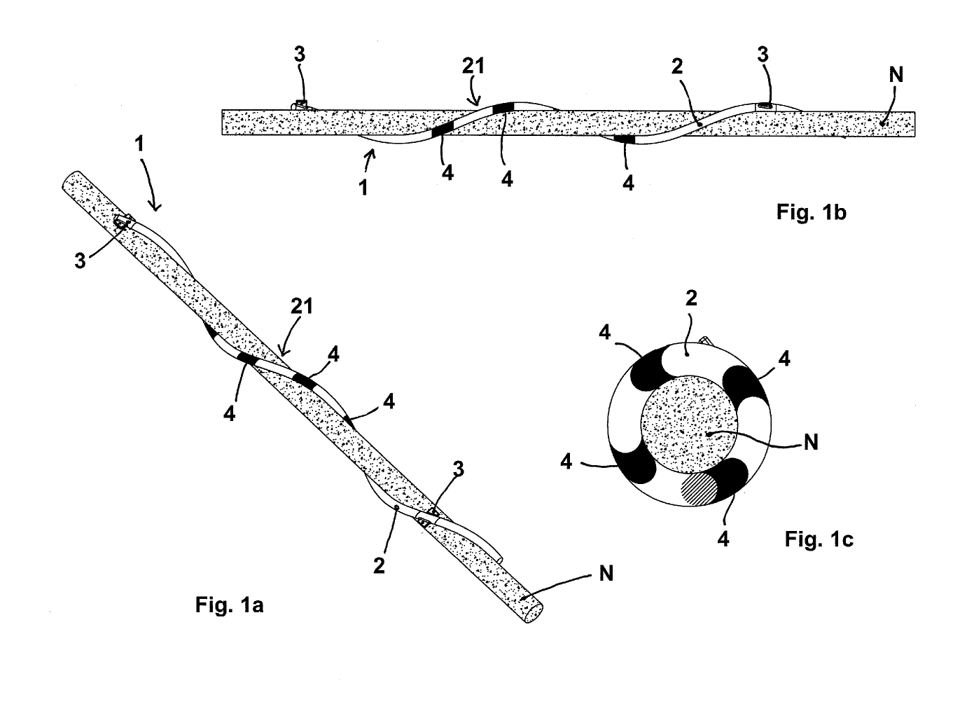

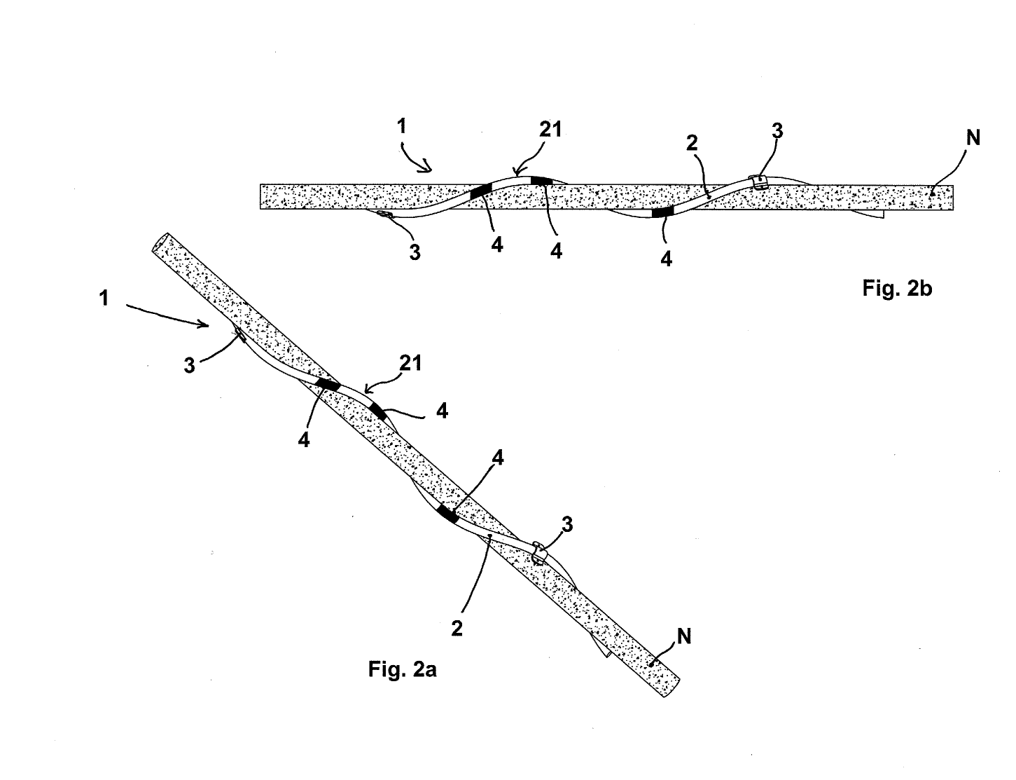

As shown in FIGS. 1a, 1b, 2a, 2b, said stimulation end (2) is essentially helical in shape and is suited to be wrapped around the nerve (N) to be treated, so that it adheres to the nerve over its entire length.

Furthermore, in order to guarantee maximum stimulation effectiveness, each one of said stimulation electrodes (4) occupies the entire cylindrical wall of a segment or section of said stimulation end (2), so as to be always in contact with said nerve (N), independently of the position of the catheter on the nerve itself.

Said stimulation end (2) is deformable, for example by traction.

If a traction force is exerted on said stimulation end (2), it can assume a helical shape with any diameter and/or pitch, according to the dimensions of the nerve (N) to be wrapped, and furthermore, once it has been wrapped around the nerve (N), it can become tight around the nerve (N) and adhere to it perfectly and completely.

Furthermore, said stimulation end (2) can also be deformed until it assumes an essentially rectilinear linear shape, depending on the profile of said nerve (N). Said configuration of the stimulation end is particularly suitable for positioning in proximity to the nerve, along its profile.

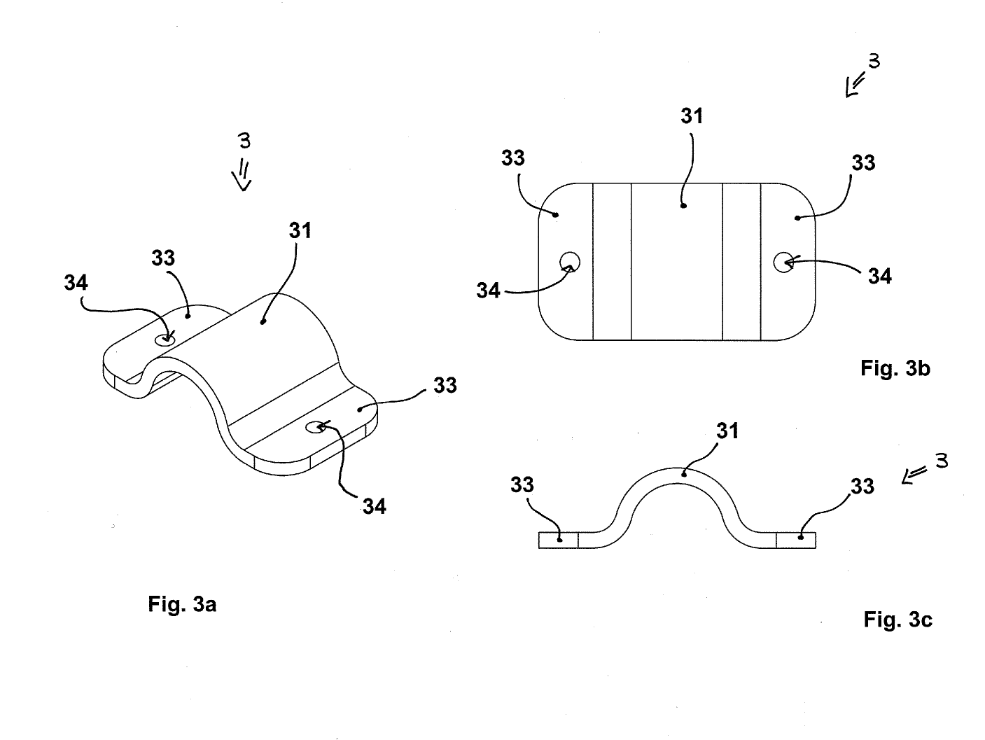

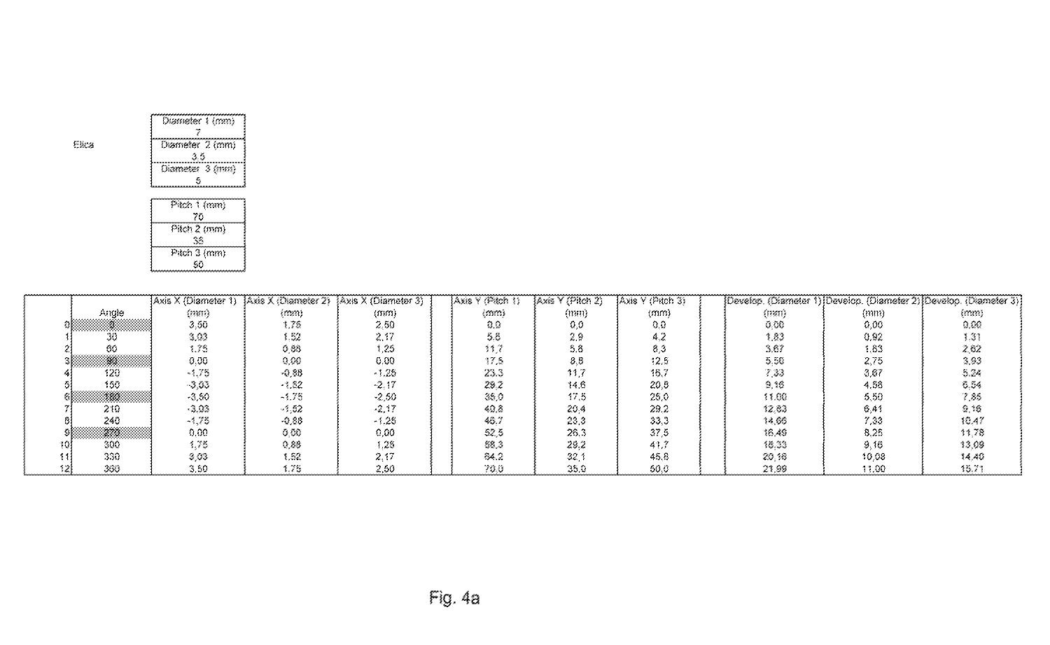

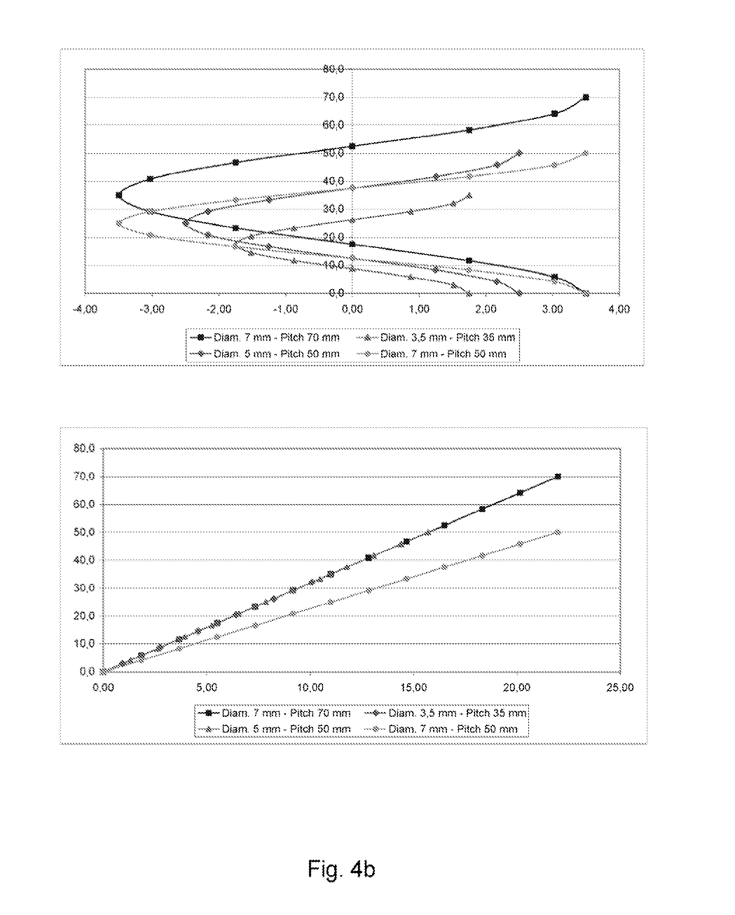

The new electro catheter (1) also comprises one or more anchorage means (3) for anchoring said stimulation end (2) to the nerve (N), an implementation of which is illustrated in FIGS. 3a, 3b and 3c.

In the preferred embodiment of the invention, said electro catheter (1) comprises at least two of said anchorage means (3), one positioned upstream and one positioned downstream of said segment (21) of said stimulation end (2) where said stimulation electrodes (4) are distributed.

Each one of said anchorage means (3) comprises a strip or portion (31) suited to at least partially wrap said stimulation end (2) and one pair of opposite tabs (33), each one with at least one hole (34) or seat suited to constrain a suture thread for fixing the electro catheter to the nerve (N).

Said anchorage means (3) can be effectively used to implant the electro catheter (1) by surgery, while they can be easily and quickly removed for the percutaneous application of said electro catheter (1) through the suitable introducer.

According to the invention, only said tabs may be removable (33), for example they can be precut in order to create a predefined breaking point.

These are the schematic characteristics that are sufficient to carry out the invention for a person skilled in the art, consequently construction changes may be made that do not affect the substance of the innovative concept disclosed herein.

Therefore, with reference to the above description and the attached drawings, the following claims are expressed.

The invention claimed is:

1. An electro catheter (1) for neuro stimulation, comprising:
a stimulation end (2) configured to be placed in proximity to a nerve (N),
wherein said stimulation end (2) has an essentially helical shape, suited to be wrapped around the nerve (N).

2. The electro catheter (1) according to claim 1, wherein said stimulation end (2) is deformable, said stimulation end having a helical shape that is variable in one or both of diameter or pitch, according to dimensions of the nerve (N) to be wrapped.

3. The electro catheter (1) according to claim 1, wherein said stimulation end (2) is variable and assume an essentially rectilinear linear shape according to a profile of said nerve (N), said stimulation end (2) being configured to be positioned in proximity to the nerve, along the profile of the nerve.

4. The electro catheter (1) according claim 1, wherein said stimulation end (2) is deformable through traction or compression.

5. The electro catheter (1) according to claim 4, wherein, after wrapping around the nerve (N), said stimulation end (2) is structured to be deformed through traction so that said stimulation end (2) becomes tight around the nerve (N) and adheres better to the nerve (N).

6. The electro catheter (1) according to claim 1, wherein said stimulation end (2) comprises one or more stimulation electrodes (4), each occupying an entire cylindrical wall of a section or segment of said stimulation end (2) and configured to come into contact with said nerve (N).

7. The electro catheter (1) according to claim 6, further comprising one or more anchorage members (3) for anchoring said stimulation end (2) to said nerve (N), each one of said anchorage members (3) comprising at least one or one pair of fixing tabs (33) configured to be constrained to the nerve (N) with a suture thread.

8. The electro catheter (1) according to claim 7, further comprising at least two of said anchorage members (3), one positioned upstream and one downstream of a segment (21) of said stimulation end (2) where said stimulation electrodes (4) are distributed.

9. The electro catheter (1) according to claim 7, wherein each one of said anchorage members (3) comprises a strip or portion (31) configured to at least partially wrap said stimulation end (2) and one pair of said opposite tabs (33), each one said opposite tabs (33) having at least one hole (34) or seat configured to constrain said suture thread for fixing the electro catheter to the nerve (N).

10. The electro catheter (1) according to claim 7, wherein said anchorage members (3) or said fixing tabs (33) are removable for percutaneous application of said electro catheter (1).

11. The electro catheter (1) according to claim 1, wherein said electro catheter is multipolar.
